(19) United States
(12) Patent Application Publication
(10) Pub. No.: US 2012/0165763 A1
(43) Pub. Date: Jun. 28, 2012

(54) EYE MEDICATION APPLICATION DEVICE

(76) Inventors: Eric Bugna, Fairfield, CA (US); Emile Vincent Bugna, San Rafael, CA (US)

(21) Appl. No.: 12/979,626

(22) Filed: Dec. 28, 2010

(51) Int. Cl. A61F 9/00

(52) U.S. Cl. 604/300

(57) ABSTRACT

A one piece eye medication application device allows the user to easily align the medication bottle with the inner corner of the eye, preventing the human reaction of blinking and keeping the medication in the eye. The device includes a one piece elongated member having an integrated ring with an opening.

### BACKGROUND OF THE INVENTION

The present invention generally relates to an eye medication application device. More specifically, the present invention relates to an eye medication application device that aligns the medication container and the eye of the user for easy delivery of the eye medication.

Dispensing medication by a person to his eye from a container often results in misses and wasted medication, since positioning the container close to the eye immediately triggers the natural human reaction to blink.

Various devices have been used to align an eye medication dispenser in relation to the eye by mechanical structures such as spacers. Unfortunately, the devices of the prior art are cumbersome, expensive, and difficult to manage, especially by older people.

As can be seen, there is a need for a simple device that allows the user to easily align the eye medication container over the eye, preventing the human reaction of blinking in order to keep the medication on the eye.

### SUMMARY OF THE INVENTION

In one aspect of the present invention, the eye medication application device includes a one piece elongated member having an integrated ring with an opening.

In another aspect of the present invention, the eye medication application device includes a one piece elongated member having an integrated ring with an opening and the elongated member includes a bend near the ring.

These and other features, aspects and advantages of the present invention will become better understood with reference to the following drawings, description and claims.

### BRIEF DESCRIPTION OF THE DRAWINGS

FIG. 1 illustrates a perspective front view of an eye medication application device according to an exemplary embodiment of the present invention;

FIG. 2 illustrates a perspective front view of the eye medication application device of FIG. 1 showing an eye medication bottle positioned on the device;

FIG. 3 illustrates a perspective front view of the eye medication application device of FIG. 1 showing the eye medication application device in a working position; and

FIG. 4 illustrates a cross sectional view of the eye medication application device of FIG. 3 showing the eye medication application device in working position.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Various inventive features are described below that can each be used independently of one another or in combination with other features.

Broadly, embodiments of the present invention generally provide a one piece eye medication application device that allows the user to easily align the medication bottle with the inner corner of the eye, preventing the human reaction of blinking and keeping the medication in the eye.

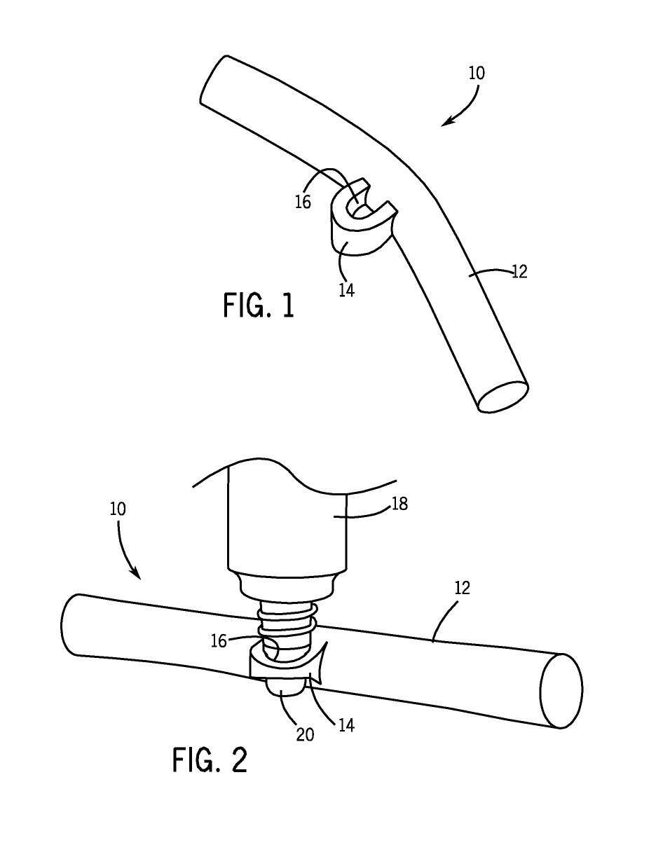

FIG. 1 illustrates an eye medication application device 10 according to an exemplary embodiment of the present invention. The eye medication application device 10 may include a one piece elongated member 12 having an integrated ring 14 with an opening 16.

The elongated member 12 may be made of a flexible material that allows the user to flex the eye medication application device 10 to concur with the contour of the inner corner area 22 of the eye of the user. In one embodiment, the elongated member 12 may be made of plastic. The elongated member 12 may be produced by injection molding. The elongated member 12 may have a cylindrical, rectangular, square, or prism shape. In one embodiment, the elongated member 12 may have a cylindrical shape. The elongated member 12 may have a length between about 4 and 10 cm. In one embodiment, the elongated member 12 may have a length of 7 cm. The elongated member 12 may have a cross section between 3 and 7 mm. In one embodiment, the elongated member may have a cross section of 7 cm.

The elongated member 12 may include a first flat side for insertion of the eye medication bottle 18 and second beveled side for resting on the user's inner corner area 22 of the eye. The elongated member 12 may bend at the point where the ring 14 is located. In one embodiment, the elongated member may bend 25 degrees at the point where the ring 14 is located.

The ring 14 may be made of the same material of the elongated member 12. The ring 14 may have a diameter between 3 to 7 mm. In one embodiment, the ring 14 may have a diameter of 5 mm.

FIG. 2 illustrates the eye medication application device 10 showing an eye medication bottle 18 positioned on the device 10. The tip 20 of the medication bottle 18 may be placed at the opening 16 of the elongated member 12. The opening 16 may be round, rectangular, square, oval, or elliptical.

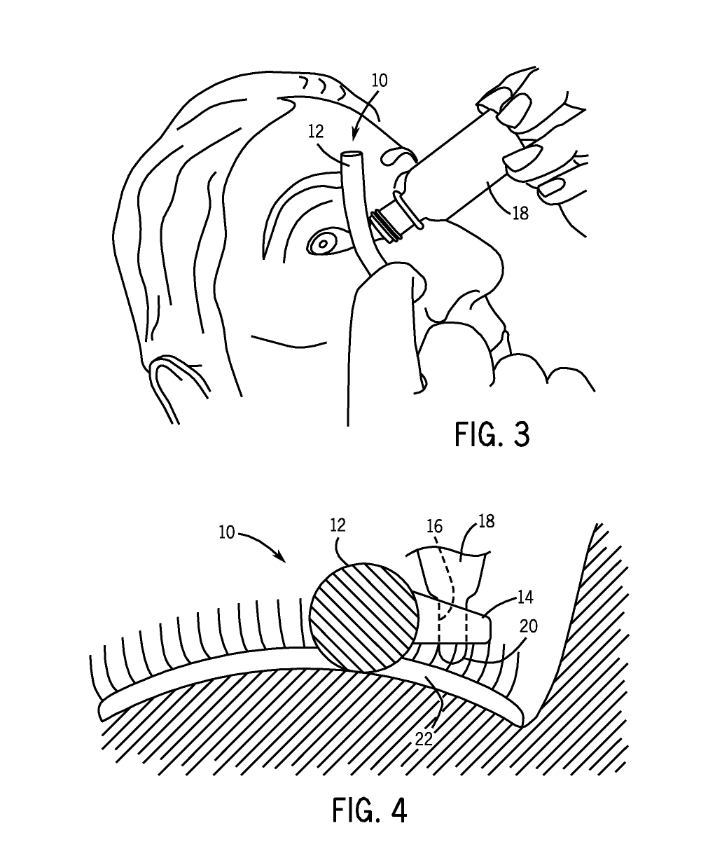

FIGS. 3 and 4 illustrate the eye medication application device 10 in a working position. The user may place the tip 20 of the medication bottle 18 on the opening 16 formed by the ring 14. The user may place the elongated member 12 at the inner corner 22 of the eye of the user. Then the elongated member 12 may extend above and below the eye and acts as a dam to keep the medication in the eye. The user may gently squeeze the eye medication bottle 18 to dispense the eye medication directly into the inner corner area 22 of the eye.

It should be understood, of course, that the foregoing relates to exemplary embodiments of the invention and that modifications may be made without departing from the spirit and scope of the invention as set forth in the following claims.

We claim:

1. An eye medication application device comprising:
an elongated member having an integrated ring with an opening.

2. The eye medication application device according to claim 1, wherein the elongated member is made of a flexible material.

3. The eye medication application device according to claim 1, wherein the elongated member has a cylindrical shape.

4. The eye medication application device according to claim 1, wherein the elongated member has a first side that is flat and a second side that is beveled.

5. An eye medication application device comprising:
an elongated member having an integrated ring with an opening;
wherein the elongated member includes a bend near the ring.

6. The eye medication application device according to claim 5, wherein the bend is a 25 degree bend.

7. The eye medication application device according to claim 5, wherein the elongated member is made of a flexible material.

8. The eye medication application device according to claim 5, wherein the elongated member has a cylindrical shape.

9. The eye medication application device according to claim 5, wherein the elongated member has a first side that is flat and a second side that is beveled.
